(19) United States
(12) Patent Application Publication
(10) Pub. No.: US 2008/0103527 A1
(43) Pub. Date: May 1, 2008

(54) FLEXIBLE ENDOSCOPIC SUTURE ANCHOR APPLIER

(76) Inventors: David T. Martin, Milford, OH (US); Michael S. Cropper, Edgewood, KY (US); Richard F. Schwemberger, Cincinnati, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/553,489

(22) Filed: Oct. 27, 2006

(51) Int. Cl. A61B 17/04

(52) U.S. Cl. 606/232

(57) ABSTRACT

A suture anchor applier comprises a needle, a flexible shaft, and flexible sleeve. The suture anchor applier is flexible enough to allow passage through and manipulation within a working channel of an articulated endoscope. The suture anchor applier is also stiff enough to resist buckling or bending when extended distally beyond the end of an endoscope and as the needle penetrates into tissue.

### BACKGROUND

The following disclosure relates to surgery, and more particularly to endoscopic surgical techniques and devices. Surgery generally refers to the diagnosis or treatment of injury, deformity, or disease. A wide variety of surgical techniques have been developed. One type of surgery is called minimally invasive surgery, which typically involves entering the body through the skin or through a body cavity or anatomical opening while minimizing damage to these structures. Minimally invasive medical procedures usually involve less operative trauma for the patient compared to open surgical procedures. Minimally invasive surgical procedures are also generally less expensive, reduces hospitalization time, causes less pain and scarring, and reduces the incidence of complications related to the surgical trauma, thus speeding the recovery.

Endoscopes are often used during minimally invasive surgical procedure to visualize the organs and structures inside the body. Endoscopes generally use a light delivery system to illuminate the tissue under inspection. Typically the light source is outside the body and the light is typically directed via an optical fiber system. Images are captured, usually through a lens system, and transmitting to a monitor. Some endoscopes include working channels through which medical instruments may be introduced into the body to biopsy or operate. Working channels can also be independent of the endoscope. Endoscopes may be rigid or flexible. Some flexible endoscopes are steerable to facilitate positioning the endoscope in the body.

Sutures are often used during surgical procedures to hold skin, internal organs, blood vessels, and other tissues in the body. A suture is typically an elongate flexible filament, but may take a variety as different thread or thread-like structures, including without limitation fibers, lines, wires, and the like. A suture may be a homogeneous or heterogeneous, and may also comprise a single filament or a composite suture, such as a two or more twisted or woven filaments. In addition, a suture may be made from a wide array of absorbable (i.e., metabolized by the body) or non-absorbable materials known in the art.

A variety of different techniques and devices have been developed to deliver and attached sutures to tissue. Some techniques involve piercing tissue with needles, tying or forming knots or loops, delivering anchors such as t-tags, x-tags and other flexible or rigid anchors, and the like. Disclosed herein are novel endoscopic delivery and attachment techniques and devices for anchoring sutures.

### BRIEF DESCRIPTION OF DRAWINGS

While the specification concludes with claims that particularly point out and distinctly claim the invention, it is believed the invention will be better understood from the following description taken in conjunction with the accompanying drawings illustrating some non-limiting examples of the invention. Unless otherwise indicated, like reference numerals identify the same elements.

FIG. 1 depicts a perspective view of a suture anchor applier;

FIG. 2 depicts a cross-sectional side view of a suture anchor applier, with a generic anchor depicted schematically;

FIG. 3 depicts a side view of an anchor;

FIG. 4 depicts a perspective view of a handle; and

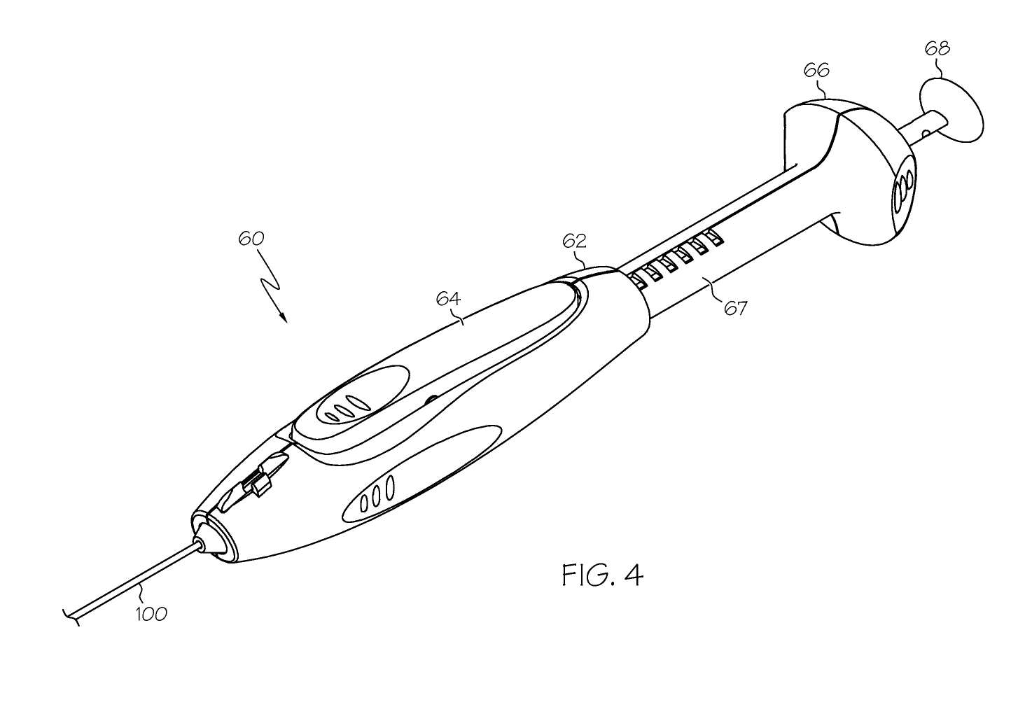

FIG. 5 depicts a schematic example of buckling.

### DETAILED DESCRIPTION

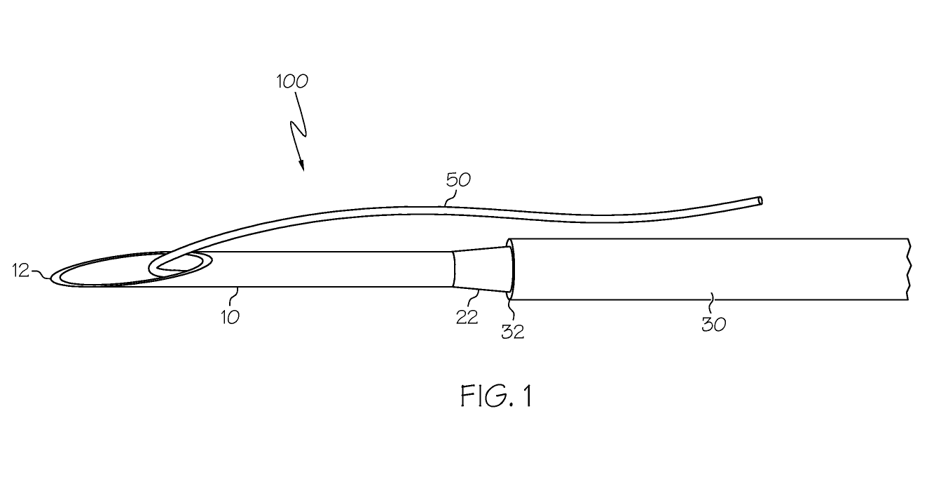

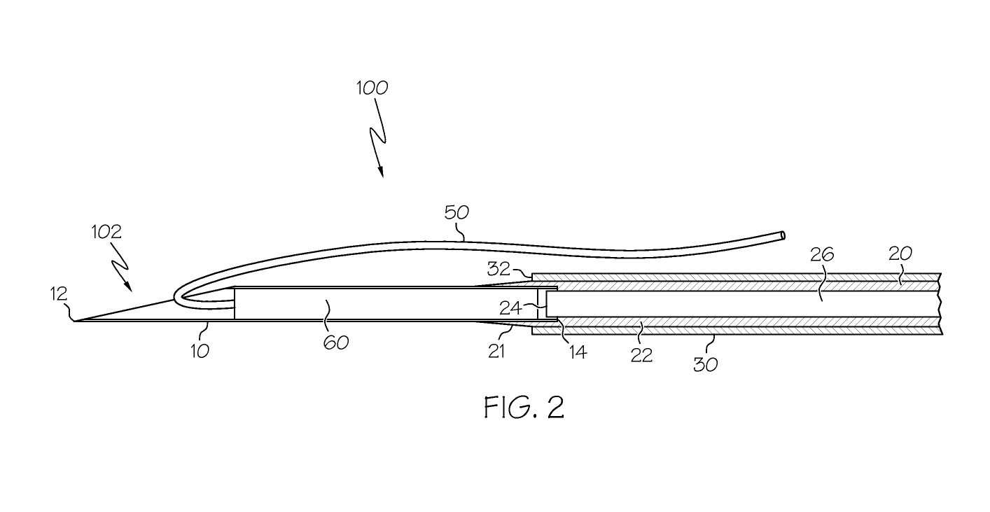

FIGS. 1 and 2 an example of a suture anchor applier. The suture anchor applier (100) comprises a needle (10), a slender flexible shaft (20), and a flexible sleeve (30).

The needle (10) has a sharp distal end (12), a proximal end (14), a length between the proximal and distal ends (12, 14). The needle (10) is hollow along its length and partially defines the cannula (102) of the anchor applier (100). The needle (10) is substantially rigid, meaning it has at least 3 times the bending rigidity of the slender flexible shaft (20). The needle (10) can be made from a variety of different materials. One suitable material is stainless steel. To facilitate the needle (10) passing through an articulated endoscope, the length of the needle (10) may be less than about ¾ inches, preferably less than 0.65 inches, and most preferably less than 0.6 inches. Optionally, the outside surface of the needle (10) can include visual indicia, such as stripes or bands of alternating colors, so the operator can determine the depth the needle (10) has penetrated tissue (10).

The slender flexible shaft (20) comprises an elongate flexible sheath (22) connected to the proximal end (14) of the needle (10) using known techniques such adhesives, interference fits, ultrasonic welding, and the like. The flexible sheath (22) is hollow along its length and partially defines the cannula (102) of the anchor applier (100). The flexible sheath (22) is dimensioned to fit in the working channel of a flexible endoscope, preferably with an outside diameter less than about 3.7 mm and a length greater than 80 cm. For other applications, such as through a trocar, the length could much shorter. The distal end of the flexible sheath (22) has a tapered geometry (21) at the interface of the needle (10). The flexible sheath (22) in this example is make from extruded PEEK; however, other structures and materials could also be used, such as a hypo-tube with selective spiral cuts, a plastic sleeve with selective spiral cuts, a braided metal wire tube, and the like. The slender flexible shaft (20) further comprises an elongate flexible push rod (26) positioned in the cannula of the flexible sheath (22). The push rod (26) may extend substantially along the length of the cannula (102). The push rod (26) is axially slidably in the flexible sheath (22) cannula. In this example the push rod (26) is made from a NITINOL wire, which may optionally be coated with a lubricant or coating such as TEFLON. Naturally, other structures and materials could also be used, such as stainless steel wire, stainless steel hypotube with spiral cuts, extruded plastic or polymeric rod or tube, such as PEEK, PEKK and nylon, and the like.

The flexible sleeve (30) is positioned around the flexible shaft (20). The flexible sleeve (30) can slide axially relative the flexible shaft (20) and needle (10) beyond the distal end (12) to prevent the needle (10) from snagging in the working channel. In addition, the flexible sleeve (30) can function as a depth limiting device by proximally retracting the leading edge (32) of the sleeve (30) to selected longitudinal position along the needle (10) or flexible shaft (20). The needle (10) can penetrate tissue up to the point of the tissue contacts the leading edge (32). The flexible sleeve (30) in this example is made from HDPE and has an outside diameter less than 3.7 mm and a wall thickness of about 0.01 to about 0.015 inches. Naturally, the flexible sleeve (30) can be made from other materials such as TEFLON, urethane, nylon, pebax, and the like.

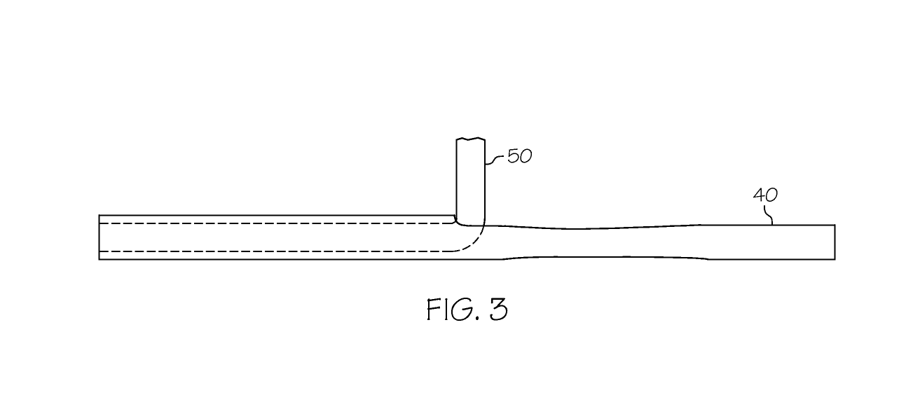

A suture anchor (60) is positioned in the cannula (102), either in the needle (10) or the flexible sheath (22), distally from the push rod (26). An interference fit between the anchor (60) and the cannula (102) may be used to provide frictional resistance to prevent the anchor (60) from discharging inadvertently. Optionally, a plurality of anchors could be positioned in tandem in the cannula (102). FIG. 3 illustrates one example of an anchor (40) with a suture (50) shown partially in phantom. The suture (50) has a deployed position, as depicted in FIG. 3, where the suture (50) extends transverse from the anchor (40). The suture (50) also has a delivery position, as shown in FIGS. 1 and 2, where the suture (50) is coextensive with or parallel to the longitudinal axis of the anchor (40). In this embodiment, the suture (50) extends out from the cannula (102) and distally from the distal end (12) of the needle (10). The anchor (40) and suture (50) are positioned in the cannula (102) in the delivery position, and implanted in tissue in the deployed position. Some non-limiting examples suitable anchors are disclose in co-owned and co-pending U.S. patent application Ser. No. 11/538,975 filed on 5 Oct. 2006, hereby incorporated by reference. Naturally, other types of suture anchors may also be used, including without limitation other types of T-tags, X-tags, expandable baskets, spring expanding anchors, umbrella anchors, barbed anchors, Christmas tree anchor, NITINOL anchors, and the like.

A handle (60) is connected to the proximal end of the suture anchor applier (100). By pushing and pulling the handle body (62) relative an endoscope working channel the suture anchor applier (100) may be advanced or retracted along the working channel. The handle (60) in this example is operatively connected to the flexible push rod (26) and the flexible sleeve (30) to control three functions: needle exposure, needle lock, and anchor deployment. The anchor actuator (68) is connected to the push rod (26) to selectively slide the push rod (26) relative the flexible sheath (22) to deploy anchors (60). Optionally, the anchor actuator (68) can be locked relative the needle exposure actuator (66) to prevent inadvertent deployment of an anchor. The needle exposure actuator (66) is connected to the flexible sleeve (30) to selectively expose the needle (10) and to control the needle penetration depth by slide the flexible sleeve (30) relative the flexible sheath (22). Indicia on the shaft (67) indicate to the operator the position of the leading end (32) of the flexible sleeve (30), which corresponds to the needle penetration depth. Rocker button (64) locks the needle exposure actuator (66) relative the body (62) thus locking the needle penetration depth. While the actuators shown here are illustrated as buttons and plungers, other types of actuators could also be used, such as knobs, levers, motors, pistons, and the like.

The following describes one exemplary use of a the suture anchor applier (100). After positioning a flexible endoscope to a desired position in a patient, the suture anchor applier (100) and suture (50) are threaded into proximal end of the working channel with the flexible sleeve (30) covering the distal end (12) of the needle (10). After pushing the suture anchor applier (100) and suture (50) the length of the working channel, the distal end (12) may be extended distally from the working channel. The leading edge (32) of the flexible sleeve (30) may be retracted thus exposing the needle (10) and setting the desired penetration depth. Typically the depth will be set for transmural or intramural penetration into the tissue. The suture anchor applier (100) is advanced distally until the needle (10) penetrated the tissue up till the leading edge (32) contacts the tissue. The push rod (26) is then actuated till the distal end (24) contacts the anchor (60) and pushes the anchor out of the cannula (102). Once ejected the anchor (60) will shift to its deployed position. As such, the suture (50) will be anchored to the tissue. The suture anchor applier (100) may then be withdrawn from the working channel leaving the suture (50) anchored in the tissue with the suture (50) in the working channel for subsequent manipulation as the surgeon may desire. For instance, two or more sutures can be anchored into tissue and cinched together using the locking devices disclosed in U.S. patent application Ser. Nos. 11/437,440 and 11/437,441 or U.S. Pat. No. 5,899,921.

The flexible components, including the flexible shaft (20) and flexible sleeve (30), of the suture anchor applier (100) balance two conflicting requirements. First, the suture anchor applier (100) is flexible enough to allow passage through and manipulation within a working channel of an articulated endoscope. Second, the suture anchor applier (100) is stiff enough to resist buckling or bending when extended distally beyond the end of an endoscope and as the needle (10) penetrates into tissue. For example, the needle (10), flexible shaft (20), and flexible sleeve (30) may be slidably inserted in and extended through a working channel of an articulated flexible endoscope with less than 4 lbs of force, and more preferably less than 2 lbs of force. As another example assume the flexible sheath (22), which comprises the composite structure of the flexible sheath (22) and push rod (26), will transfer the majority of the axial load to the needle (10) during penetration. In this example the flexible shaft (20) may have sufficient rigidity to distally extend two inches out of an endoscope working channel and impart an axial load on the needle (10) of at least 1 lb force without buckling.

As one with ordinary skill in the art will recognize, buckling is a failure mode characterized by a sudden failure by bending of a structural member that is subjected to compressive axial load where the actual compressive stresses at failure are bigger than the ultimate compressive stresses that the material is capable of withstanding. This mode of failure is also described as failure due to elastic instability. The buckling maximum load, sometimes called the critical load, causes a column to be in a state of unstable equilibrium, that is, any increase in the loads or the introduction of lateral force will cause the column to fail by buckling.

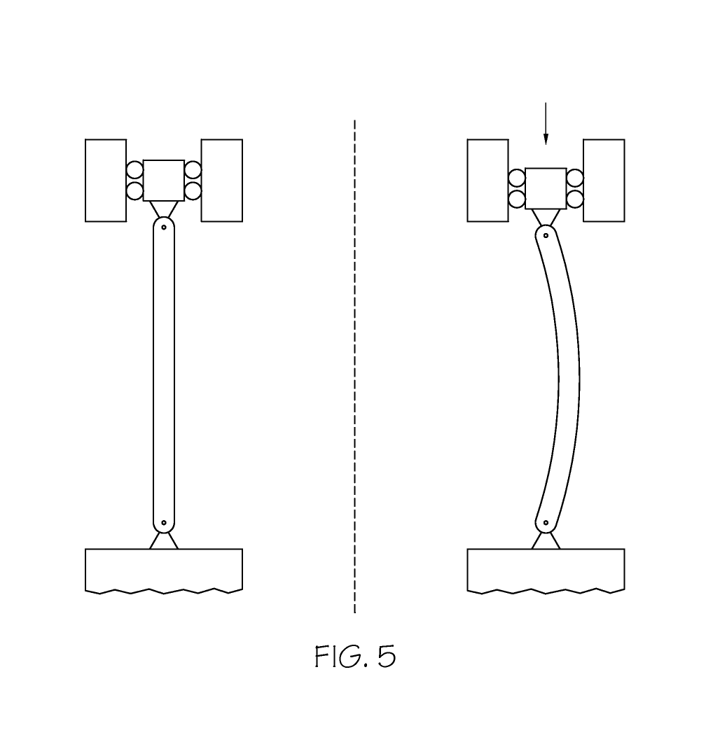

FIG. 5 illustrates a schematic example of a column under an axial load exhibiting the characteristic deformation of buckling. In this illustration the ends of the column are free to turn, which in this example is illustrated as having pinned ends. The left frame depict the column prior to an axial load being introduced to the column, and the right frame depicts the buckled column after the buckling maximum load was introduced.

In one embodiment, the two inch length of the flexible shaft (20), comprising the composite structure of the flexible sheath (22) and push rod (26), adjacent the needle (10) has a buckling maximum load greater than about ½ lbs and less than about 5 lbs when measured with free ends. More preferably, the two inch length of the flexible shaft (20) has a buckling maximum load less than about 1½ lbs when measured with free ends.

The 18th-century mathematician Leonhard Euler derived a formula which gives the maximum axial load that a long, slender ideal column can carry without buckling. An ideal column is one which is perfectly straight, homogeneous, and free from initial stress. Euler's equation for column buckling of a column is shown in equation 1.

$$P = \frac{K {π}^{2} EI}{{L}^{2}} \qquad ( Eq . 1 )$$

where:
P is the buckling maximum load;
K is a constant whose value depends upon the conditions of end support of the column. For both ends free to turn K=1, for both ends fixed K=4, for one end free to turn and the other end fixed K=2 approximately, and for one end fixed and the other end free to move laterally K=¼;
E is the Modulus of Elasticity of the material;
I is the area moment of inertia of the column; and
L is the length of the column.

For purposes of illustration, we will assume the flexible sleeve (30) has minimal contribution to resisting bucking and that the flexible shaft (20) is a slender ideal column having free ends, so K=1. While the flexible shaft (20) is not an ideal column, nor will it in use have free ends, the buckling characteristics of the flexible shaft (20) can nevertheless be understood in view of the relationship of the variables in Euler's equation.

Most needles that would be used in an endoscopic setting have a penetration force below 1 lb. If it is assumed that the length of needle exposed to buckling outside of the endoscope is approximately 2 inches and that the needle is 0.6 inches long, then the flexible length of the flexible shaft (20) exposed to buckling is 1.4 inches. The EI term in Equation 1 thus should be larger than 0.2 lb-in² in order to achieve a buckling strength greater than 1 lbf. For the specific case of the flexible shaft (20) structure described above, this EI term can be modeled as the sum of the EI values of the PEEK flexible sheath (22) and the NITINOL push rod (26). The sum of the EI values of the PEEK flexible sheath (22) and the NITINOL push rod (26) is between 0.8 and 1.0 lb-in² yield buckling strengths above 1 lbf. Naturally, this desired EI value will vary depending on the amount of needle exposed outside the endoscope, the length of the needle, and the fit between the two parts of the assembly.

The EI term also relates to the stiffness of the flexible shaft (20). Equation 2 shows the equation for deflection of a cantilever beam.

$$v = \frac{{PL}^{3}}{3 EI} \qquad ( Eq . 2 )$$

where:
P is the force applied to the end of the beam;
v is the deflection at the end of the beam;
E is the Modulus of Elasticity of the material;
I is the area moment of inertia of the column; and
L is the length of the column.

While this equation does directly relate to the ability to pass a device through an articulated endoscope, it does provide insight into the factors that may be important. One with ordinary skill in the art will recognize that a shaft that exhibited low deflection in response to an applied force would be relatively stiff and thus difficult to pass through an articulated endoscope. Conversely, a shaft that exhibited high deflections in response to the same applied force should be relatively flexible and thus easier to pass through an articulated endoscope. It is the EI term that provides the stiffness component of this equation. Thus, as the EI term associated with the dimensions and material of the shaft increase, so should the force required to insert the device through an articulated endoscope. 1.1 lb-in² value is a good guideline for the maximum flexible shaft (20) stiffness that will allow insertion through an articulated endoscope.

Based on this assessment, an appropriate EI value for the flexible shaft (20) should be between 0.8 and 1.1 lb-in², and is preferred to be on the low end of this range. Note that this assessment applies several simplifying assumptions, but should provide a way of estimating the performance of a design.

Preferably, the foregoing devices will be processed before surgery. First, a new or used device is obtained and if necessary cleaned. The device can then be sterilized. In one sterilization technique, the device is placed in a closed and sealed container, such as a plastic or TYVEK bag. The container and device are then placed in a field of radiation that can penetrate the container, such as gamma radiation, x-rays, or high-energy electrons. The radiation kills bacteria on the device and in the container. The sterilized device can then be stored in the sterile container. The sealed container keeps the device sterile until it is opened in the medical facility.

Having shown and described various embodiments and examples, further adaptations of the methods and apparatuses described herein can be accomplished by appropriate modifications by one of ordinary skill in the art without departing from the scope of the present invention. Several of such potential modifications have been mentioned, and others will be apparent to those skilled in the art. For instance, the specific dimensions and assumptions described above and scales depicted in the figures will be understood to be non-limiting examples. Accordingly, the scope of the present invention should be considered in terms of the following claims and is understood not to be limited to the details of structure, materials, or acts shown and described in the specification and figures.

1. A surgical instrument, comprising
a) a needle with a sharp distal end, a proximal end, a length between the proximal and distal ends less than about ¾ inches, and a cannula extending along the needle length;
b) a slender flexible shaft having an outside diameter of less than about 3.7 mm, the slender flexible shaft comprising:
(i) an elongate flexible sheath comprising a distal end connected to the proximal end of the needle, the flexible sheath further comprising a cannula aligned with the needle cannula;
(ii) an elongate flexible push rod positioned in the cannula of the flexible sheath and axially slidably in the sheath cannula, the push rob being operably connected to an actuator;
wherein the 2 inch length of the flexible shaft adjacent the needle has a buckling maximum load greater than about ½ lbs and less than about 5 lbs when measured with free ends; and
c) a suture anchor positioned in the needle cannula or sheath cannula.

2. The surgical instrument of claim 1, wherein the buckling maximum load of the needle with free ends is at least three times the buckling maximum load of the flexible portion.

3. The surgical instrument of claim 1, further comprising a depth limiting sleeve slidably receiving the flexible shaft.

4. The surgical instrument of claim 3, wherein the depth limiting sleeve is operably connected to an actuator.

5. The surgical instrument of claim 1, wherein the needle is rigid.

6. The surgical instrument of claim 1, wherein the flexible shaft is at least 80 cm in length.

7. The surgical instrument of claim 1, wherein the slender flexible shaft has an EI value between about 0.8 and about 1.1 lb-in².

8. A method of processing a device for surgery, comprising:
a) obtaining the surgical instrument of claim 1;
b) sterilizing the surgical instrument; and
c) storing the surgical instrument in a sterile container.

9. A flexible endoscopic suture anchor applier, comprising:
a) a hollow needle less than about ¾ inches in length and dimensioned to receive a suture anchor;
b) a slender flexible shaft dimensioned to be inserted into a working channel of an articulated flexible endoscope, the flexible shaft comprising an elongate sheath and a push rod axially slidable in the elongate sheath, the flexible shaft being characterized by a 2 inch length of the flexible shaft has a buckling maximum load less than about 1½ lbs when measured with free ends.

10. The flexible endoscopic suture anchor applier of claim 9, wherein the slender flexible shaft has an EI value between about 0.8 and about 1.1 lb-in².

11. The flexible endoscopic suture anchor applier of claim 9, further comprising a depth limiting sleeve slidably receiving the slender flexible shaft.

12. A method of processing a device for surgery, comprising:
a) obtaining the flexible endoscopic suture anchor applier of claim 9;
b) sterilizing the flexible endoscopic suture anchor applier; and
c) storing the flexible endoscopic suture anchor applier in a sterile container.

13. A flexible endoscopic suture anchor applier, comprising:
a) a needle comprising a sharp distal end, a proximal end, and a length between the distal and proximal ends;
b) a slender flexible sheath comprising a distal end connected to the needle proximal end, a proximal end, and a length between the distal and proximal ends;
c) a cannula extending between the needle distal end and the sheath proximal end; and
d) an slender flexible push rod positioned substantially along the length of the cannula, the push rod being capable of sliding axially in the cannula;
e) a suture anchor positioned in the cannula and distally from the push rod;
f) a flexible sleeve slidably receiving the flexible shaft and needle;
g) a handle operatively connected to the flexible push rod and flexible sleeve;
wherein the needle, flexible sheath, and flexible sleeve can be slidably inserted in and extended through a working channel of an articulated flexible endoscope with less than 4 lbs of force, and
wherein composite structure of the flexible sheath and push rod have sufficient rigidity to distally extend two inches out of an endoscope working channel and impart an axial load on the needle of at least 1 lb force without buckling.

14. The flexible endoscopic suture anchor applier of claim 13, further comprising a suture connected to the anchor.

15. The flexible endoscopic suture anchor applier of claim 14, wherein the suture extend out from the cannula and distally from the distal end of the needle.

16. The flexible endoscopic suture anchor applier of claim 13, wherein the needle length is less than about 0.6 inches.

17. The flexible endoscopic suture anchor applier of claim 13, wherein the sheath length is greater than about 80 cm.

18. The flexible endoscopic suture anchor applier of claim 17, wherein the flexible sleeve has an outside diameter less than about 3.7 mm.

19. The flexible endoscopic suture anchor applier of claim 9, wherein the composite structure of the flexible sheath and push rod has an EI value between about 0.8 and about 1.1 lb-in².

20. A method of processing a device for surgery, comprising:
a) obtaining the flexible endoscopic suture anchor applier of claim 13;
b) sterilizing the flexible endoscopic suture anchor applier; and
c) storing the flexible endoscopic suture anchor applier in a sterile container.
